(19) United States
(12) Patent Application Publication
(10) Pub. No.: US 2018/0144574 A9
(43) Pub. Date: May 24, 2018

(54) AUTOMATED OPHTHALMIC LENS AND SOLUTIONS DISPENSING UNIT

(71) Applicants: Franca Renna, Jacksonville, FL (US); Randall Pugh, Jacksonville, FL (US); Daniel Wang, Jacksonville, FL (US); Francis E. Mirmina, Jacksonville, FL (US)

(72) Inventors: Franca Renna, Jacksonville, FL (US); Randall Pugh, Jacksonville, FL (US); Daniel Wang, Jacksonville, FL (US); Francis E. Mirmina, Jacksonville, FL (US)

(21) Appl. No.: 12/700,945

(22) Filed: Feb. 5, 2010

Related U.S. Application Data
(60) Provisional application No. 61/236,658, filed on Aug. 25, 2009.
(60) Provisional application No. 61/171,593, filed on Apr. 22, 2009.

(51) Int. Cl. G07F 11/00; G07F 17/00; G07F 9/02; G16H 40/20; G06Q 10/08; G06Q 50/22

(52) U.S. Cl. CPC G07F 11/00; G07F 17/0092; G06Q 50/22; G16H 40/20; G06Q 10/087; G07F 9/026

(57) ABSTRACT

The present invention discloses methods and apparatus for dispensing ophthalmic lenses and solutions and replenishing those lenses physically or virtually dispensed. In addition, in some embodiments, a patient may prepare an order for lenses and solutions dispensed.

### CROSS REFERENCE

The present invention claims priority to U.S. Provisional Patent Application Ser. No. 61/236,658 filed on Aug. 25, 2009 and entitled, “Automated Ophthalmic Lens Dispensing Unit”, which is relied upon and incorporated herein by reference.

### FIELD OF USE

The present invention provides for automated apparatus for dispensing ophthalmic lenses or solutions stored in the practice or available on the internet, tracking which lenses are dispensed and replenishing the apparatus with additional lenses or solutions.

### BACKGROUND

Eye care practitioners (sometimes referred to herein as an “ECP” or multiple “ECPs” have traditionally maintained a physical or online/internet-based inventory of solutions or trial ophthalmic lenses, whether in hydrated form or stored as zero-diluent (dry lenses), to fit on patients following an examination. An ophthalmic lens, such as a contact lens provides a predetermined optical quality. Today's technologies provide for a large number of various qualities that an ophthalmic lens may possess. For example, a contact lens may provide one or more vision correction functions, as well as cosmetic enhancement and therapeutic effects. Each function is provided by a physical characteristic of the lens.

Basically, a design incorporating a refractive quality into a lens provides vision corrective functionality. A pigment incorporated into the lens can provide a cosmetic enhancement. An active agent incorporated into a lens can provide a therapeutic functionality. Each combination of functions may be embodied in a separate SKU number and consequently, an ECP may need to stock and track usage of a large number of ophthalmic lens SKUs online or in the practice to measure what is utilized by the ECP's office.

Similarly, various types of ophthalmic lens solutions may be stocked in an ECP office and require tracking.

Generally, an ECP's office is poorly equipped to track an inventory of lenses and reorder new lenses if a number of lenses and solutions in stock reaches a minimum threshold optimal for that office. Typically the task is allocated to a sales representative for a lens manufacturer. However, sales representatives only visit ECP offices on a periodic basis, and during the intervening time periods, an inventory may run out of some SKUs. In addition, a physical or online, internet-based inventory that is manually maintained by a human being is inefficient and cumbersome. It requires that a sales representative maintain an inventory of lenses and solutions and attempt to anticipate usage by the ECP offices that the sales representative will make a site visit to.

### SUMMARY

Accordingly, the present invention includes an apparatus for maintaining a inventory of one or both of ophthalmic lenses and ophthalmic solutions and track dispensing of the lenses and solutions, replenishment of lens SKUs dispensed and in some embodiments, order placement for ophthalmic lens types or solutions dispensed for trial by a patient and worn a trial basis by the patient.

### DESCRIPTION OF THE DRAWINGS

FIG. 1 illustrates a network diagram of an Automated Dispensing Unit in digital communication with a distributed network and user access device.

FIG. 2 illustrates aspects diagram of some additional aspects of the present invention.

FIG. 3 illustrates additional method steps useful in some implementations of the present invention.

FIG. 4 illustrates method steps according to some embodiments of the present invention.

FIG. 5 illustrates method steps according to some additional aspects of the present invention.

FIG. 6 illustrates a processor that may be used to implement some embodiments of the present invention.

### DETAILED DESCRIPTION OF THE INVENTION

The present invention includes methods and apparatus for tracking one or both of physical and online (internet-based) dispensing of ophthalmic lenses from an eye care practitioner's office. In addition, the present invention includes method and apparatus for displaying information related to a dispensed lens and placing an order for one or more ophthalmic lenses based upon a lens dispensed.

### GLOSSARY

In this description and claims directed to the presented invention, various terms may be used for which the following definitions will apply:

“Automatic Dispensing Unit” or “ADU”; as used herein refers to automated apparatus that is functional to order and/or dispense an ophthalmic lens and track distribution of an ophthalmic lens based upon a description of the lens; and automatically track which lenses are stocked and sold within the apparatus an available for sale online or dispensing.

“Lens” as used herein, refers to any ophthalmic device that resides in or on the eye. These devices can provide optical correction or cosmetic enhancement. For example, the term lens can refer to a contact lens, intraocular lens, overlay lens, ocular insert, optical insert or other similar device through which vision is corrected or modified, or through which eye physiology is cosmetically enhanced (e.g. iris color, limbal ring) without impeding vision. In some embodiments, the preferred lenses of the invention are soft contact lenses made from silicone elastomers or hydrogels, which include but are not limited to silicone hydrogels, and fluorohydrogels.

“Ophthalmic Solution” as used herein refers to a solution is in conjunction with an ophthalmic lens for one or both of cleaning and storing the ophthalmic lens.

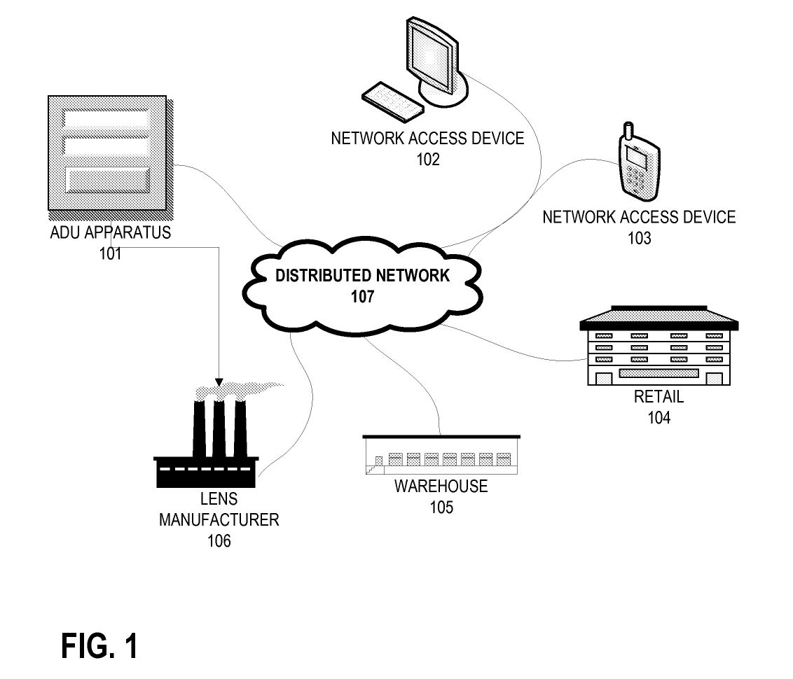

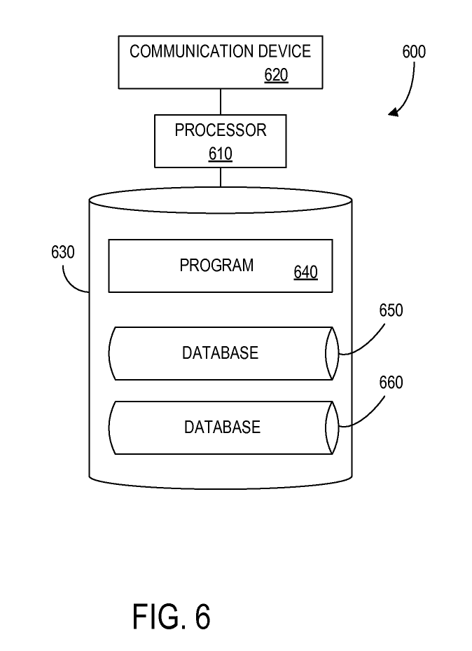

Referring now to FIG. 1, a network diagram illustrates how an ADU 101 may be connected via a distributed network 107, such as, for example, the Internet, a cellular link, a virtual private network or other vehicle for transferring digital data. The ADU Automated apparatus may include a machine such as those described in the U.S. patent application Ser. No. 12/639,291 and entitled, “An Apparatus and Method for Distributing Ophthalmic Lenses”, which is relied upon and incorporated herein by reference. In addition, the apparatus may include a programmable processor and storage device connected to a human readable display device, as discussed further below in regard to FIG. 6.

Generally, the ADU 101 is functional to track an inventory accessible to, or inherent within, the ADU 101 and to dispense one or more ophthalmic lenses upon demand. In some preferred embodiments, the ADU 101 is stocked with ophthalmic lenses via a single package or cartridge refill unit which contains multiple lens stock keeping units (“SKUs”) denoting different lens characteristics. As lenses are dispensed, digital data descriptive of the dispensing is generated. The digital data may be processed by storing the data within the ADU 101 or transmitted to a destination 102-106 connected to the distributed network 107.

A destination 102-106 can include, for example, a network access device 102 that includes a display and keyboard accessing the distributed network 107 under the control of a processor. The network access device may therefore include a personal computer, mobile device, laptop or terminal. Other network access devices 103 may be mobile in nature and include personal digital assistants (“PDA”s), cellular phones with network access capabilities, net books, or other relatively small processor run appliances with the ability to send and receive digital data across a network.

Still other embodiments include network access devices 104-105 associated with a retail location for ophthalmic lenses and solutions, a warehouse for ophthalmic lenses and solutions and an ophthalmic lens and solutions manufacturing facility.

The ADU 101 may therefore receive ophthalmic lenses and solutions into an inventory and dispense the inventoried ophthalmic lenses and solutions at an eye care practitioner (“ECP”) location or other monitored location. Digital data descriptive of which lenses are present in an ADU 101 inventory, and which lenses are dispensed, is transmitted from the ADU 101 and thereby available to users with access to a network access device 102-106. The digital data descriptive of lenses present in the ADU 101 inventory and which lenses are dispensed may be transmitted one or both of: with no artificial delay introduced (real time), or on a periodic basis. In some embodiments, the ADU 101 compiles a report descriptive of an aggregate of lenses dispensed and transmits the report to network access devices associated with related entity. The related entity can be, for example, a lens manufacturer, a market analyst, an ophthalmic lens retailer; an ophthalmic lens warehouse, or other interested entity.

In some embodiments, an ADU 101 may be functional to dispense one or more trial lenses and solutions to a patient and also initiate an order for commercially available lenses. Initiation of an order may include transmission of digital data descriptive of the patient and lenses and solutions to be shipped to the patient. The digital information initiating may therefore include, for example, any or all of the following: patient name and address, billing information, payment information, lens SKU, quantity of lenses, ECP providing prescription for the lenses, and any other information generally received by an online retail site for lens sales. One exemplary online sales vehicle includes Acuvue-Direct™ which processes orders for ophthalmic lenses via a distributed network 107 which includes the Internet.

In another aspect, an ADU 101 may be functional to dispense one or more trial lenses and solutions to a patient and also initiate an automated reminder, such as ACUMINDER™, to replace the patient's contact lenses. A schedule may commence based upon the type of lens dispensed and a suggested lens wearing schedule. In some embodiments, the ADU 101 may include executable code that is programmed to receive a start date and a period of lens wear. The ADU may then automatically transmit a reminder, such as, for example, via one or more of: email, text message, social media, RSS, and telephone. The reminder can include a human interpretable message that instructs them to do a specific action, such as replace their lenses. Additional embodiments can include an automated apparatus connected to an internet access device 102-106 which can transmit a reminder to a patient when it is time to replace their lenses. One example of an online mechanism includes ACUMINDER™. The online mechanism can be initiated by a transmission from the ADU indicating a start date of lens wear and suggested time of wear for a lens SKU dispensed.

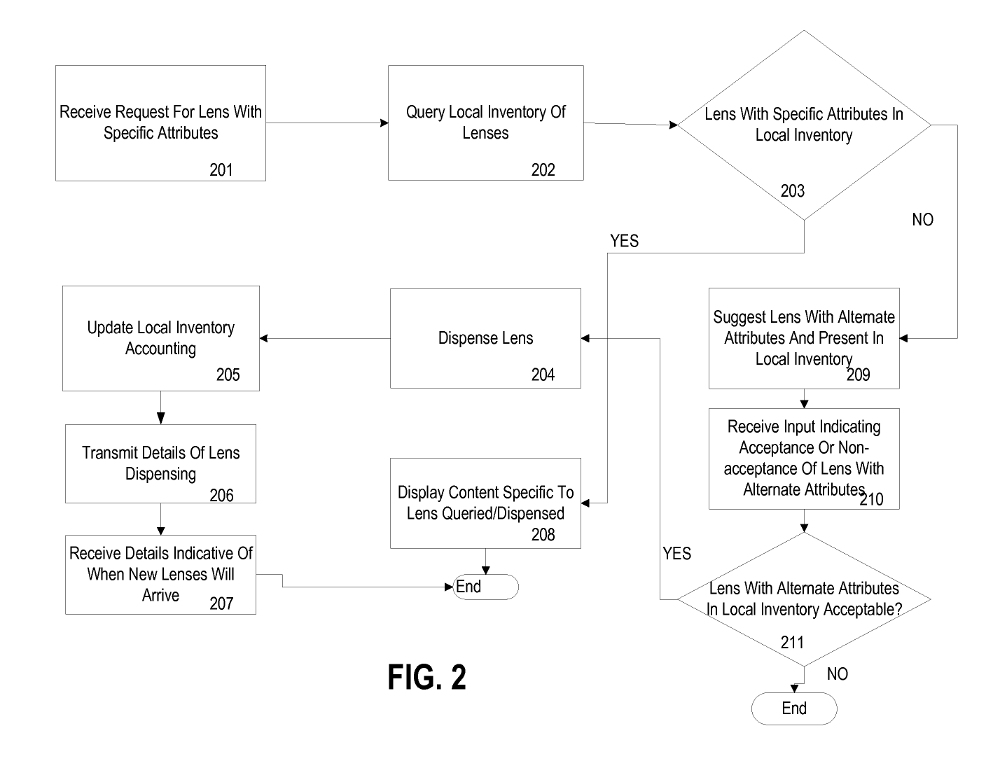

Referring now to FIG. 2, a block diagram of functions performed by automated apparatus according to the present invention is illustrated. At 201 a request may be received for an ophthalmic lens with specific attributes. Attributes may include, for example, characteristics of the lens, such as, a lens diameter, lens power, lens material, suggested lens replacement schedule and other lens parameters. In some embodiments, attributes may include a specific brand or line of lenses and solutions.

The ADU 101 may automatically query an inventory of lenses accessible to the ADU 101. In some embodiments, the inventory will be inherent to the ADU and in other embodiments the inventory may be linked via automation to an external inventory. At 203, digital data is generated indicating whether a lens with the specific attributes is available online and/or in a local inventory. At 204, if a lens with the specific attributes is available online and/or in a local inventory, the lens is dispensed to the patient's shipping address (e.g. home) and/or at 205 the local inventory is updated. At 209, if a lens with the specific attributes is not available in inventory, an alternate lens may be suggested. The alternate lens may, for example, include a lens with similar attributes and which is available in inventory, can be suggested via a human readable interface.

At 210, the ADU may receive an input of whether the lens with alternate attributes online or in local inventory is acceptable. The indication may include, for example, digital data generated by a user interface. At 211, if the input indicates the lens is acceptable, the alternate lens is dispensed 204 and the local inventory is updated 205. At 206, details of the lens and solution dispensing are transmitted. The transmission may be digital data descriptive of which lenses and how many of each lens have been dispensed and via a distributed network 107 to a remote or local destination and accessible via a network access device 102-106.

In some embodiments, the transmission from the ADU 101 can be received, for example, by an eCommerce interface, a warehouse 105, or a lens manufacturer 106 and be utilized to generate an ADU 101 reload order. In some preferred embodiments, the reload order can be based upon dispensing trends by the ADU 101. Additional factors that a reload order can be based upon can include a lens and solution manufacturer new product release, a product promotion, warehouse inventory, patient demographics or other criteria.

In another aspect, in some embodiments, a retail facility may also receive digital data transmitted by the ADU 101. The digital data received can be descriptive of which lenses across the competitive contact lens industry are dispensed to which patients. The retail facility 104 may use the data to queue up an order for patients which includes the same type of lens as the lenses dispensed. In some specific embodiments, office management software, such as OfficeMate may be utilized to process an order. A patient may access the retail facility 104 and complete processing of a queued up order, all based upon data generated by the ADU 101.

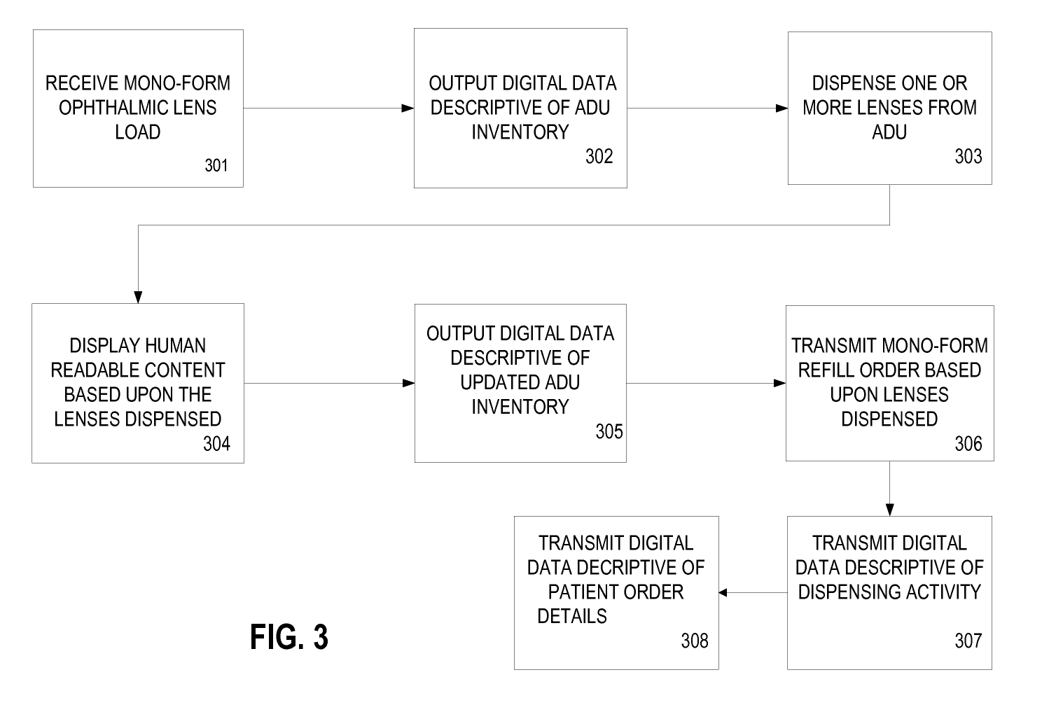

Referring now to FIG. 3, at 301, an ADU 101 may receive a mono-form ophthalmic lens load. A mono-form load is more fully includes a single cartridge or other package with multiple different ophthalmic lenses. In some embodiments, the multiple lenses can include multiple SKUs. A user simply installs the mono-form load and the ADU 101 determines which lenses have been loaded. The ADU 101 can also be programmably functional to combine a list of loaded lenses with lenses already in inventory. The aggregate of the loaded lenses and the lenses already in inventory becomes a current list of lenses in inventory. At 302, the ADU outputs digital data descriptive of a current ADU 101 inventory.

At 303, the ADU 101 dispenses one or more lenses from the ADU 101. At 304, in some embodiments, the ADU can display human readable content based upon the lenses dispensed. The human readable content may include, for example, features of the lens dispensed that an ECP may wish to discuss with a patient who will trial dispensed lenses. Other content may include pricing, sales information, or alternative lenses. For example, a lens may be dispensed which is formed from a first material with a suggested wear modality and the displayed content may recommend a second lens of a different material or qualities and perhaps a different wear modality. Still other content may include suggestions relating to proper care for the lens, or complimentary products, such as a suggested solution for cleaning and/or storing the dispensed lenses. Almost any pertinent data may be displayed, wherein the concept that the human readable content will be specifically related to the one or more lenses dispensed.

At 305, the ADU 101 may output digital data descriptive of an updated ADU 101 inventory, following dispensing of lenses from the ADU 101. At 306, the ADU 101 transmits a mono-form refill order based upon which lenses are dispensed. In addition, to a one-for-one replacement, a refill order may take into consideration trends in which lenses are being dispensed, including for example, lens material, lens brand, lens optical attributes, or other characteristics.

At 307, the ADU may additionally transmit digital data descriptive of dispensing activity unlimited to multiple competitive product brands. As discussed above, data descriptive of dispensing activity may assist factories determine manufacturing schedules for new lenses, inventory levels for a manufacturer, ordering levels for a distributor, inventory levels for a distributor, pricing adjustments, ordering of base materials used to manufacture additional lenses, or other purposes related to the business of manufacturing and selling ophthalmic lenses and solutions.

At 308, in some embodiments, a user interface may be presented on the ADU 101 which permits a patient to order additional lenses from a manufacturer or a distributor or a retail location. In some embodiments, an existing web portal may be used to front end input into an ordering system. One example of a web portal includes the ecommerce site known as ACUVUE® DIRECT™, which may be found on the internet at http://www.acuvue.com/acuvue-direct.htm. The ADU may provide an interface which queues up information related to a patient and also to lenses and solutions that have been dispensed to the patient.

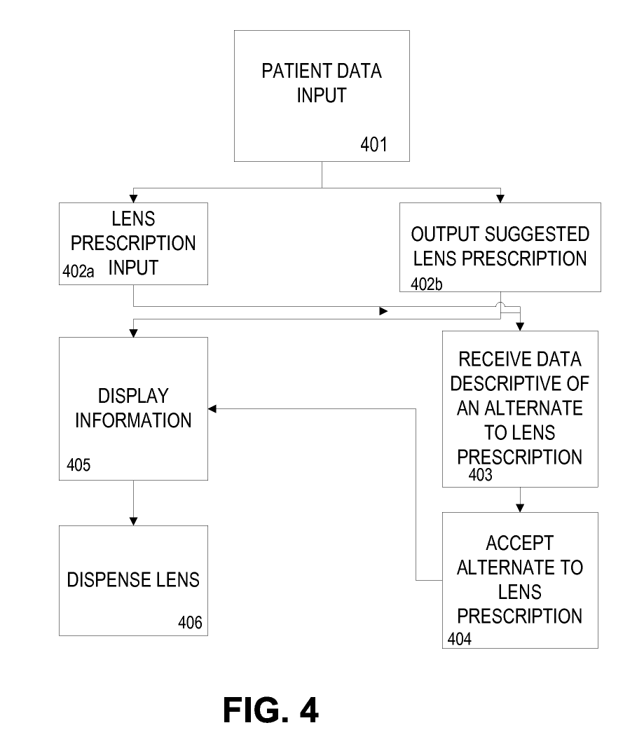

Referring now to FIG. 4, in another aspect, an ADU according to the present invention may be operative to assist an ECP manage which lenses are trialed by offering alternatives to a particular patient. At 401, patient data is input into the ADU 101. The patient data may include, for example, data identifying the patient and insurance or other payment options available to the patient. The patient data may also include data descriptive of one or more variables pertaining to one or both of a patient's eyes.

Patient variables, can include, for example one or more of: metrics of a patients eye; a specific need, conditions of use; a symptom; a medical condition, patient's medical history, online social behaviors or other variable is input into a computerized system to determine a modification of a formed lens or a lens design. In one example a wavefront can be determined for an eye via an abberometer, such as a COAS or Wavescan device. This wavefront can capture residual optical errors that either a standard lens or fitting lens is not correcting for.

At 402a the ADU 101 has a lens prescription input. In such instances, the lens prescription can be determined by an ECP office using techniques well known and established and drawing upon an ECP office member's knowledge of products ophthalmic lens products and solutions available.

At 402b, The ADU can receive input descriptive of patient biometrics, biofeedback (e.g. bionic glucose level measurements) and eye measurements. The ADU 101 may then output a suggested lens prescription based upon the patient data.

At 403, whether a lens prescription is input 402a or suggested by the ADU 402b, the ADU may also present alternative lens choices. The alternative lens choices may be based upon one or more of: lenses available in the ADU; lens or solution product performance; marketing promotions or other business objective, all of which may be captured in a simple algorithm and included in executable software utilized to operate the ADU 101.

At 404, a user input may be received which accepts an alternate lens or proceed with an input lens 402a or suggested lens 402b. At 405, the ADU may display human readable data related to lenses dispensed, and at 406, selected lenses and solutions are dispensed.

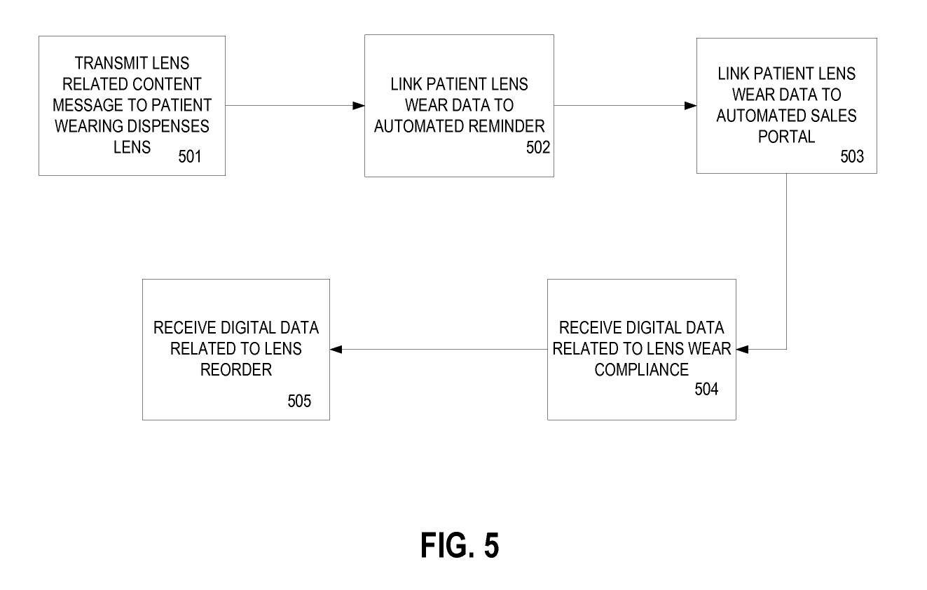

Referring now to FIG. 5, according to the present invention, an ADU 101 may also be programmed to support user needs subsequent to dispensing lenses for the patient. At 501, the ADU 101 may transmit digital data including one or more lens related content to a user device associated with a patient wearing dispensed lenses. Transmission may include, for example one or more of: email message, text message, social media messaging, telephone call or other electronic communication. At 502 the ADU may also link digital data descriptive of a patient's lens wearing practices to an automated reminder of when a patient should change out their lenses.

At 503, patient lens wear and solutions data may also be transmitted out to an automated lens and solutions sales portal, such as the ACUVUE® DIRECT™ website or other distributor affiliate accessible via the Internet. At 504 the ADU 101 may receive back digital data related to a patient's lens wear compliance and at 505 the ADU 101 may receive back digital data related to a patient's lens and solutions ordering activities.

Referring now to FIG. 6 a controller 600 is illustrated that may be used in some embodiments of the present invention. The controller 600 includes a processor 610, which may include one or more processor components coupled to a communication device 620. In some embodiments, a controller 600 can be used to transmit energy or information data to the Energy Source placed in the ophthalmic lens.

The controller can include one or more processors, coupled to a communication device configured to communicate energy or information via a communication channel. The communication device may be used to electronically transmit and receive digital data related to the functions discussed above.

The communication device 620 may also be used to communicate, for example, with one or more human readable display devices, such as, for example: an LCD panel, a LED display or other display device or printer. In some preferred a touch screen is utilized for human interface with the ADU 101.

The processor 610 is also in communication with a storage device 630. The storage device 630 may comprise any appropriate information storage device, including combinations of magnetic storage devices (e.g., magnetic tape, radio frequency tags, and hard disk drives), optical storage devices, and/or semiconductor memory devices such as Random Access Memory (RAM) devices and Read-Only Memory (ROM) devices.

The storage device 630 can store a program 640 for controlling the processor 610. The processor 610 performs instructions of the program 640, and thereby operates in accordance with the present invention. For example, the processor 610 may receive information descriptive of lens dispensing, lens inventory and the like. The storage device 630 can also store patient related data in one or more databases 650 and 660.

### CONCLUSION

In the sections above, detailed descriptions of some embodiments of the invention have been given. The description of both preferred and alternative embodiments are exemplary embodiments only, and it is understood that to those skilled in the art that variations, modifications and alterations may be apparent. It is therefore to be understood that said exemplary embodiments do not limit the scope of the underlying invention.

The present invention, as described above and as further defined by the claims below, provides apparatus and methods for automated dispensing of lenses.

What is claimed is:

1. Apparatus for dispensing ophthalmic lenses, the apparatus comprising:
an automated dispensing device capable of dispensing an ophthalmic lens in response to a digital signal;
a computer processor in digital communication with the automated dispensing device;
a digital media storage device in communication with the computer processor and storing executable software code which is executable upon demand and operative with processor and the automated dispensing device to:
store digital data comprising a record descriptive of an inventory of ophthalmic lenses in an automated dispensing device;
receive a digital data input descriptive of one or more lenses dispensed from the automated dispensing device; and
update the record descriptive of an inventory of ophthalmic based upon the digital data input descriptive of one or more lenses dispensed.

2. The apparatus of claim 1 additionally comprising a communications device linking the computer processor to a distributed network, wherein the executable software code is additionally operative to transmit data descriptive of a current inventory in the automated dispensing device.

3. The apparatus of claim 1 additionally comprising a communications device linking the computer processor to a distributed network, wherein the executable software code is additionally operative to transmit data descriptive of a patient to whom a lens was dispensed from the automated dispensing device.

4. The apparatus of claim 1 additionally comprising a communications device linking the computer processor to a distributed network, wherein the executable software code is additionally operative to transmit data descriptive of a report comprising an aggregate of lenses dispensed and transmits the report to a network access device associated with a related entity.

5. The apparatus of claim 1 additionally comprising a communications device linking the computer processor to a distributed network, wherein the executable software code is additionally operative to generate a mono-form refill order and transmit data descriptive of the mono-form refill order to network access device associated with a related entity.

6. The apparatus of claim 5 wherein the related entity comprises a manufacturer of ophthalmic lenses.

7. The apparatus of claim 5 wherein the related entity comprises a distributor of ophthalmic lenses.

8. The apparatus of claim 1 additionally comprising a user interface for receiving input descriptive of a patient and a lens modality and the software is additionally operative to calculate an ophthalmic lens wear schedule and transmit a reminder to the patient comprising the ophthalmic lens wear schedule.

9. The apparatus of claim 1 additionally comprising a user interface capable of displaying human readable content and the software is additionally operative with the processor for the processor to receive digital data descriptive of a lens dispensed and retrieve data from the storage related to the lens dispensed and display the data retrieved on the user interface.

10. The apparatus of claim 1 additionally comprising a user interface capable of displaying human readable content and a communications device linking the computer processor to a distributed network and wherein the software is additionally operative with the processor for the processor to receive digital data descriptive of a lens dispensed and retrieve data via the distributed network, wherein the retrieved data is related to the lens dispensed and display content on the user interface based upon the retrieved data.

11. The apparatus of claim 1 additionally comprising a user interface capable of displaying human readable content and a communications device linking the computer processor to a distributed network and wherein the software is additionally operative with the processor for the processor to transmit digital data descriptive of a lens dispensed and receive data via the distributed network, wherein the received data comprises a shipping schedule for lenses to replace the lenses dispensed.

12. The apparatus of claim 1 additionally comprising a user interface capable of displaying human readable content and a communications device linking the computer processor to a distributed network and wherein the software is additionally operative with the processor for the processor to receive digital data descriptive of a lens dispensed and transmit data via the distributed network to a network device associated with a patient for whom the lens was dispensed and wherein the transmitted data is related to the lens dispensed.

13. The apparatus of claim 1 additionally comprising a user interface capable of displaying human readable content and wherein the software is additionally operative with the processor for the processor to receive digital data descriptive of an ophthalmic lens and display information related to the ophthalmic lens.

14. A computerized method of dispensing ophthalmic lenses, the method comprising;
establishing a connection between an automated ophthalmic lens dispensing unit in an eye care practitioner office and a distributed network, wherein the connection is suitable for communication of digital data;
storing a record comprising digital data descriptive of inventory comprising ophthalmic lenses located in the automated ophthalmic lens dispensing unit;
dispensing one or more ophthalmic lenses from the automated ophthalmic lens dispensing unit;
updating the record comprising digital data descriptive of an inventory based upon the one or more lenses dispensed; and
transmitting digital data descriptive of the inventory.

15. The method of claim 14 additionally comprising the step of transmitting digital data to a network access device associated with a patient, wherein the digital data comprises human readable content related to an ophthalmic lens dispensed to the patient.

16. The method of claim 14 additionally comprising the step of generating a wear schedule for an ophthalmic lens and transmitting digital data to a network access device associated with a patient, wherein the digital data comprises human readable content related to the wear schedule for the ophthalmic lens dispensed to the patient.

17. The method of claim 14 additionally comprising the step of generating a proposed order for ophthalmic lenses of a same type as a type of lens dispensed and transmitting digital data to a network access device associated with a patient, wherein the digital data comprises human readable content related to the wear proposed order for ophthalmic lenses of the same type dispensed to the patient.

18. The method of claim 14 additionally comprising the step of transmitting digital data descriptive of a lens dispensed and a patient to a web portal capable of receiving an order for ophthalmic lenses of a same type as the lens dispensed.

19. The method of claim 18 additionally comprising the step of receiving digital data descriptive of an order for ophthalmic lenses for the patient.

20. The method of claim 19 additionally comprising the step of transmitting digital data comprising a prescription for ophthalmic lenses and solutions from a person licensed to prescribe, wherein the prescription comprises a lens type and optical qualities.
